(19) United States
(12) Patent Application Publication
(10) Pub. No.: US 2010/0233156 A1
(43) Pub. Date: Sep. 16, 2010

(54) Treating Central Nervous System Injury with Beta and Gamma Secretase Inhibitors

(75) Inventors: Mark P. Burns, Kensington, MD (US); G. William Rebeck, Washington, DC (US); David J. Loane, Washington, DC (US)

Correspondence Address: FISH & RICHARDSON P.C., P.O BOX 1022, Minneapolis, MN, 55440-1022, US

(73) Assignee: GEORGETOWN UNIVERSITY, Washington, DC (US)

(21) Appl. No.: 12/719,282

(22) Filed: Mar. 8, 2010

Related U.S. Application Data
(60) Provisional application No. 61/158,508, filed on Mar. 9, 2009.

(51) Int. Cl. A61K 39/395; A61K 31/216; A61K 31/55; A61P 25/00

(52) U.S. Cl. 424/130.1; 514/539; 514/212.07

(57) ABSTRACT

Provided herein are methods of treating an acute central nervous system injury in a subject. The methods comprise administering to the subject a γ or β secretase inhibitor, an Aβ antibody, or an immunogenic fragment of Aβ.

### CROSS-REFERENCE TO RELATED APPLICATION

This application claims priority to U.S. Provisional Application No. 61/158,508, filed Mar. 9, 2009, which is incorporated by reference herein in its entirety.

### STATEMENT REGARDING FEDERALLY FUNDED RESEARCH

This invention was made with government support under Grant Nos. R03NS57635 and R24HD050845 from the National Institutes of Health. The United States government has certain rights in this invention.

### BACKGROUND

Traumatic Brain Injury (TBI), one type of acute central nervous system injury, is the leading cause of mortality and disability among young individuals in developed countries, and globally the incidence of TBI is rising sharply. TBI is a disease process, with an initial injury that induces numerous biochemical and cellular changes that contribute to continuing neuronal damage and cell death over time. This continuing damage is known as secondary injury, and multiple apoptotic and inflammatory pathways are activated as part of this process. TBI is a major risk factor for the development of Alzheimer's disease, and post-mortem studies show that 30% of TBI fatalities have Aβ deposits.

### SUMMARY

Provided are methods of treating an acute central nervous system injury. Specifically, the methods comprise administering to the subject a γ or β secretase inhibitor. The γ or β secretase inhibitor is administered to the subject within 72 hours of acute central nervous system injury.

Also provided are methods of treating an acute central nervous system injury in a subject comprising administering to the subject an amyloid-β (Aβ) antibody. The Aβ antibody is administered to the subject within 72 hours of acute central nervous system injury.

Provided are methods of treating an acute central nervous system injury in a subject comprising administering to the subject an immunogenic fragment of Aβ. The immunogenic fragment of Aβ is administered to the subject within 24 hours of acute central nervous system injury.

### DESCRIPTION OF DRAWINGS

FIG. 1 is a bar graph showing that levels of Aβₓ₋₄₀ peak three (3) days following controlled cortical contusion injury (CCI). (*P<0.05, ***P<0.001 versus sham control by analysis of variance (ANOVA), Neuman-Keuls post-hoc test. Data are means±s.e.m.; n=4).

FIG. 2 is an image of a Western blot demonstrating expression of amyloid-related proteins APP, sAPP-a, BACE1, and presenilin-1 one (1), three (3), and seven (7) days after TBI in nontransgenic mice. Actin expression is shown as a control. The blot is representative of three separate experiments.

FIG. 3 is a behavioral profile of the controlled cortical impact model of murine TBI. FIG. 3a is a bar graph showing TBI causes deficits in fine motor coordination as measured by the beamwalk test. FIG. 3b is a bar graph showing deficits in coordination are not the result of deficits in gross motor function as ambulation in an open field paradigm remains unaltered. FIG. 3c is a graph showing TBI causes spatial memory deficits as measured by escape latency. FIG. 3d is a graph showing TBI causes spatial memory deficits as assessed by probe trials assessing % time in correct quadrant (left) and number of entries into correct quadrant (right). FIG. 3e is a graph showing hippocampal impairments were not due to motor deficits as swim speeds of TBI and sham injured animals were identical. FIG. 3f is a graph showing similar times were recorded when a visible platform was placed in the maze.

FIG. 4 is a bar graph showing a fine motor coordination test of Bace1^{+/+} mice and Bace1^{-/-} mice on a beam walk apparatus. Deficits in coordination are recorded as foot faults. (*P<0.05, **P<0.01, ***P<0.001 by ANOVA, Neuman-Keuls post-hoc test. Data are means±s.e.m.; n=8).

FIG. 5 is a graph showing spatial learning Bace1^{+/+} mice, Bace1^{+/+} mice after TBI, and Bace1^{-/-} mice after TBI in a Morris water maze. Each trial is the average of four individual tests, and four trials were performed with the escape latency to the platform being recorded. (**P<0.01 versus uninjured Bace1^{+/+} and ++P<0.01 versus injured Bace1^{+/+} by ANOVA, Neuman-Keuls post-hoc test. Data are means+s.e.m., n=5 for Bace1^{+/+} and n=8 for TBI groups).

FIG. 6 shows MRI images from Bace1^{+/+} and Bace1^{-/-} mice twenty-one (21) days after TBI. Representative images from three different mice in each group. Lesions on the left hemisphere appear white on the images.

FIG. 7 shows representative images of H&E stained sections from Bace1^{+/+} and Bace1^{-/-} mice 21 days after TBI, highlighting hippocampal sparing in injured Bace1^{-/-} mice.

FIG. 8 is a graph showing that Bace1^{+/+} mice have less hippocampal volume than Bace1^{-/-} mice 21 days after TBI as assessed by comparing tissue remaining in the ipsilateral side with the contralateral hippocampus. (**P<0.01 by Mann-Whitney U test, n=5).

FIG. 9 is a bar graph showing that Bace1^{+/+} mice have more lesion volume 21 days after TBI compared to Bace1^{-/-} mice. (*P<0.05 by Student's t test; data are means±s.e.m., n=5).

FIG. 10 is a bar graph showing TBI induced impairments in fine motor coordination on a beamwalk test in both vehicle and DAPT treated mice. Recovery is improved in DAPT treated mice after 21 days (***P<0.001 by ANOVA, Newman-Keuls post-hoc test. Data are means±s.e.m., n=11 for vehicle and n=8 for DAPT).

FIG. 11 is a graph showing TBI caused learning impairments in vehicle treated mice as assessed by the Morris water maze test. DAPT treatment prevents these deficits. (*P<0.05, **P<0.01, ***P<0.001 versus vehicle sham by ANOVA, Newman-Keuls post-hoc test. Data are means±s.e.m., n=6 for sham groups, n=11 for vehicle-treated TBI mice, and n=8 for DAPT-treated TBI mice.

FIG. 12 is a bar graph showing swim speed measures from the water maze test in FIG. 11.

FIG. 13 shows MRI images demonstrating the depth of lesion volume in 21 day controlled cortical impact (CCI) injured mice. The upper panel shows a vehicle treated mouse with the lesion visible through 8 consecutive 0.5 mm slices (region of hyperintensity in the upper left region of the brain). The lower panel shows the extent of injury in a chronically treated DAPT mouse. Representative images shown, n=6.

FIG. 14 shows representative H&E-stained sections of vehicle- and DAPT-treated TBI mice demonstrating the extent of tissue sparing in DAPT-treated mice. Subcortical white matter tracts are spared in DAPT-treated mice (arrowhead).

FIG. 15 is a bar graph showing lesion volume was reduced in DAPT-treated TBI mice as compared to vehicle control. (**P<0.01, Student's t test. Data are means±s.e.m., n=4).

FIG. 16 shows representative images of NeuN-stained CA1 demonstrating neuronal damage in mice after TBI. Neurons in vehicle TBI mice were shrunken and dystrophic compared to both sham control and DAPT-treated TBI neurons.

FIG. 17 is a bar graph of stereology cell counts showing that vehicle treated TBI mice have reduced neuronal populations in the ipsilateral (ipsi) CA1 region of the hippocampus compared to the contralateral (contra) CA1. This reduction is prevented in the DAPT-treated TBI mice (*P<0.05 and +P<0.05 by ANOVA, Newman-Keuls post-hoc test. Data are means±s.e.m., n=3).

FIG. 18 is a bar graph showing that BACE inhibitor IV blocks the TBI-induced increase in Aβ. Intracerebroventricular (ICV) injection of vehicle increased Aβ levels compared to non-injected (sham) controls, with TBI further increased Aβ. Post-trauma injection of BACE inhibitor IV attenuates the trauma and icy induced increases in Aβ (P<0.01 by ANOVA, Newman-Keuls post-hoc test. Data are means±s.e.m., n=3).

FIG. 19 is a bar graph showing that central administration of anti-Aβ antibody attenuates the TBI-induced increase in Aβ.

### DETAILED DESCRIPTION

### I. Gamma (γ) and Beta (β) Secretase Inhibitors

Provided herein is a method of treating an acute central nervous system injury in a subject comprising administering to the subject a γ or β secretase inhibitor, wherein the γ or β secretase inhibitor is administered to the subject at least once within about 72 hours of acute central nervous system injury. The β or γ secretase inhibitor can be administered to the subject within about 3 days (e.g., 0 to 3 days) of a central nervous system injury. As used herein 0 days refers to the time of the central nervous system injury. Optionally, the γ or β secretase inhibitor is administered on the day of the injury (e.g., within 24 hours of the injury). Optionally, the γ or β secretase inhibitor is administered within 1 hour of the injury. Optionally, the γ or β secretase inhibitor is administered within 15 minutes of the injury. The γ or β secretase inhibitor can, for example, be administered to the subject at least once daily for 21 days or less. Optionally, the γ or β secretase inhibitor is administered to the subject at least twice daily. Optionally, the γ or β secretase inhibitor is administered for at least 3 days.

Optionally, the methods further comprise selecting a subject with a central nervous system injury or a subject at risk for a central nervous system injury. A central nervous system injury can, for example, be selected from the group consisting of a traumatic brain injury, a cerebrovascular event, a spinal cord injury, and a central nervous system surgery. A subject at risk of developing a central nervous system injury can be genetically predisposed to a central nervous system injury, e.g., have a family history or have a mutation in a gene that causes the a central nervous system injury (e.g., mutations in Amiloride-sensitive epithelial sodium channels (aENaC) and prothrombic mutations). Further, a subject at risk of developing a central nervous system injury can be occupationally predisposed to a central nervous system injury, e.g., soldiers, boxers, football players, and other occupations where the risk of trauma to the head is increased. It should be noted that for subjects at risk for central nervous system injury, they may benefit from prophylactic and/or long-term administration of the γ or β secretase inhibitor. A subject currently with a central nervous system injury has one or more than one symptom of the central nervous system injury and may have been diagnosed with the acute central nervous system injury by one of skill in the art.

Further provided is an auto-injector for emergency treatment of an acute central nervous system injury in a subject, wherein the auto-injector comprises a γ or β secretase inhibitor. Emergency treatment for an acute central nervous system injury can occur when a subject has been in an accident (e.g., a vehicle-related accident (e.g., automobile, train, boat), a sports-related accident (e.g., football, basketball, hockey), and a work-related accident (e.g. soldier, construction worker)). Optionally, the auto-injector comprises a dose of 0.1-100 mg/kg body weight. Also provided is a kit (e.g. a first aid kit, a paramedic kit, or a battlefield kit) comprising the auto-injector described above.

Provided herein are methods of treating an acute central nervous system injury in a subject. Such methods include administering an effective amount of a γ or β secretase inhibitor. Optionally, a γ or β secretase inhibitor can comprise a small molecule, a polypeptide, a nucleic acid molecule, a peptidomimetic or a combination thereof Optionally, the small molecules, polypeptides, nucleic acid molecules, and/or peptidomimetics are contained within a pharmaceutical composition.

A. Small Molecules/Drugs

The γ or β secretase inhibitor can, for example, be a small molecule. The γ secretase inhibitor can, for example, be N-[N-(3,5-Difluorophenacetyl)-L-alanyl]-S-phenylglycine t-butyl ester (DAPT) or a derivative thereof Optionally, the γ secretase inhibitor can be LY450139 or a derivative thereof Optionally, the γ secretase inhibitor can be R-flubiprofen (tarenfluibil); 1367, LDDN-9918, MK-0752, and GSI-136. Optionally, the β secretase can, for example, be BACE inhibitor IV or a derivative thereof. Optionally, the β secretase inhibitor can be OM99-2, GRL-8234, GSK188909, and CTS-21166. γ and β secretase inhibitors are known and have been described in, for example, Ghosh et al., Neurotherapeutics 5:399-408 (2008); Ghosh et al., Curr. Alzheimer Res. 5:121-31 (2008); and Wolfe, Neurotherapeutics 5:391-8 (2008), which are incorporated herein by reference.

B. Inhibitory Peptides

Inhibitors of one of the subunits of the γ secretase complex or β secretase include inhibitory polypeptides. As used herein, the terms peptide, polypeptide, protein or peptide portion are used broadly herein to mean two or more amino acids linked by a peptide bond. Protein, peptide, and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more.

Inhibitory peptides also include dominant negative mutants of one of the subunits of the γ secretase complex or β secretase. Dominant negative mutations (also called antimorphic mutations) have an altered phenotype that acts antagonistically to the wild-type or normal protein. Thus, dominant negative mutants of one of the subunits of the γ secretase complex or β secretase act to inhibit the activity of the normal γ secretase complex or β secretase. Such mutants can be generated, for example, by site directed mutagenesis or random mutagenesis. Proteins with a dominant negative phenotype can be screened using methods known to those of skill in the art, for example, by phage display.

Nucleic acids that encode the aforementioned polypeptide sequences are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein. A wide variety of expression systems may be used to produce polypeptides as well as fragments, isoforms, and variants. Such polypeptides are selected based on their ability to bind one of the subunits of the γ secretase complex or β secretase and inhibit the γ secretase complex or β secretase or based on their ability to block the γ or β secretase ligand.

C. Inhibitory Nucleic Acids

Also provided herein are functional nucleic acids that inhibit expression of γ or β secretase. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of one of the subunits of the γ secretase complex or β secretase.

Functional nucleic acids are nucleic acid molecules that have a specific function, such as binding a target molecule or catalyzing a specific reaction. Functional nucleic acid molecules can interact with any macromolecule, such as DNA, RNA, polypeptides, or carbohydrate chains. Thus, functional nucleic acids can interact with the mRNA, genomic DNA, or polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation.

Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855, 5,877,022, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Col.), Glen Research (Sterling, Va.), MWB Biotech (Esbersberg, Germany), Proligo (Boulder, Col.), and Qiagen (Vento, The Netherlands). siRNA can also be synthesized in vitro using kits such as Ambion's SILENCER® siRNA Construction Kit

(Ambion, Austin, Tex.).

D. Antibodies

Proteins that inhibit one of the subunits of the γ secretase complex or β secretase also include antibodies with antagonistic or inhibitory properties. Such antibodies are preferably antibodies that bind to one of the subunits of the γ secretase complex or β secretase itself. In addition to intact immunoglobulin molecules, fragments, chimeras, single-chain antibodies, or polymers of immunoglobulin molecules are also useful in the methods taught herein, as long as they are chosen for their ability to inhibit one of the subunits of the γ secretase complex or β secretase. The antibodies can be tested for their desired activity using in vitro assays, or by analogous methods, after which their in vivo therapeutic or prophylactic activities can be are assessed.

The term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein,

Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

Digestion of antibodies to produce fragments thereof, e.g., Fab fragments, can be accomplished using routine techniques. For instance, digestion can be performed using papain. Examples of papain digestion are described in WO 94/29348 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods include site-specific mutagenesis of the nucleic acid encoding the antibody or antibody fragment. (Zoller, M. J. Curr. Opin. Biotechnol. 3:348-354, 1992).

As used throughout, antibody fragments include Fv, Fab, Fab′, or other antigen binding portion of an antibody.

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### II. Aβ Antibody

Also provided herein is a method of treating an acute central nervous system injury in a subject comprising administering to a subject an Aβ antibody, wherein the Aβ antibody is administered to the subject at least once within about 72 hours of acute central nervous system injury. The Aβ antibody can be administered to the subject within about 3 days (e.g. 0 to 3 days) of a central nervous system injury. As used herein 0 days refers to the time of the central nervous system injury. Optionally, the Aβ antibody is administered on the day of the injury (e.g. within 24 hours of the injury). Optionally, the Aβ antibody is administered within 1 hour of the injury. Optionally, the Aβ antibody is administered within 15 minutes of the injury. The Aβ antibody can, for example, be administered to the subject at least once daily for 21 days or less. Optionally, the Aβ antibody is administered to the subject at least twice daily. Optionally, the Aβ antibody is administered for at least 3 days. As described above for g or b secretase inhibitors, the antibodies can be used prophylatically and/or long term for subjects at risk of developing a central nervous system injury. All information discussed above relating to antibodies can be applied to the administration of Aβ antibodies for the inhibition of Aβ. Aβ antibodies are known and have been described, for example, in U.S. Pat. No. 7,320,790, which is incorporated herein by reference.

### III. Immunogenic Fragment of Aβ

Further provided herein is a method of treating an acute central nervous system injury in a subject comprising administering to a subject an immunogenic fragment of Aβ, wherein the immunogenic fragment of Aβ is administered to the subject within 24 hours of acute central nervous system injury. An immunogenic fragment of Aβ can, for example, include Aβ1-15 (SEQ ID NO:1). Immunogenic fragments of Aβ are known and have been described, for example, in Maier et al., J. Neurosci. 26:4717-28 (2006) and Moretto et al., J. Biol. Chem. 282:11436-45 (2007), which are incorporated herein by reference. The immunogenic fragment administered to the subject results in antibody production by the subject. Thus, administration of the immunogenic fragment of Aβ would have a similar effect to administration of the Aβ antibody itself, except for the time delay necessary for mounting an immunogenic response. Thus, the immunogenic fragment should be administered before or as soon after injury as possible. Such therapy would also be useful for subjects at risk of developing a central nervous system injury using prophylactic and/or long-term administration.

### IV. Compositions

Provided herein are compositions containing the provided small molecules, polypeptides, nucleic acid molecules, and/or peptidomimetics and a pharmaceutically acceptable carrier described herein. The herein provided compositions are suitable of administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intraventricularly, intracistemally, subdurally, intraperitoneally, intramuscularly, subcutaneously, intracavity, transdermally, intracranially, or by inhalation.

When the compositions are administered systemically, a blood brain barrier permeabilizer can be optionally included to facilitate entry into the brain. Blood brain barrier permeabilizers are known in the art and include, by way of example, bradykinin and the bradykinin agonists described in U.S. Pat. Nos. 5,686,416; 5,506,206 and 5,268,164 (such as NH₂-arginine-proline-hydroxyproxyproline-glycine-thienylalanine-serine-proline-4-Me-tyrosine.ψ(—CH2NH)-arginine-COOH).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable. Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retorviruses, Cold Spring Harbor Laboratory Press (1997), which is incorporated by reference herein for the vectors and methods of making them. The construction of replication-defective adenoviruses has been described (Berkner et al., J. virology 61:1213-20 (1987); Massie et al., Mol. Cell. Biol. 6:2872-83 (1986); Haj-Ahmad et al., J. virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived fromt eh structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrance fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. (β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1a promoter, and the retroviral long terminal repeat (LTR).

The vectors also can include, for example, origins of replication and/or markers. A marker gene can confer a selectable phenotype, e.g., antibiotic resistance, on a cell.

The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or Flag™ tag (Kodak; New haven, Conn.) sequences typically are expressed as a fusion with the encoded polypeptide. Such tags can be inserted anywhere within the polypeptide including at either the carboxyl or amino terminus.

The methods and agents as described herein are useful for both prophylactic and therapeutic treatment. For prophylactic use, a therapeutically effective amount of the agents described herein are administered to a subject prior to onset (e.g., before obvious signs of an acute central nervous system injury) or during onset (e.g., upon initial signs and symptoms of an acute central nervous system injury). Prophylactic administration can occur for several days to years prior to or in the absence of the manifestation of symptoms of an acute central nervous system injury. Prophylactic administration can be used, for example, in the preventative treatment of subjects diagnosed with a genetic predisposition to an acute central nervous system injury or prior to central nervous system surgery or trauma. Therapeutic treatment involves administering to a subject a therapeutically effective amount of the agents described herein after diagnosis or development of an acute central nervous system injury. For example, in a subject wherein the central nervous system injury is a central nervous system surgery, the γ or β secretase inhibitor or Aβ antibody can be administered before, during, or after surgery or is administered in any combination of before, during, or after surgery.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. For example, DAPT, LY450139, or a derivate thereof can be administered in a dose of 1-500 mg/kg. Optionally, the dose can be 5-250 mg/kg. Optionally, the dose can be 10-100 mg/kg. Optionally, the dose can be 25-50 mg/kg. DAPT, LY450139, or a derivate thereof can, for example, be administered to the subject up to twice daily. Optionally, DAPT, LY450139, or a derivate thereof can be administered once daily. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of injury, the extent of the injury, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products.

As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with an acute central nervous system injury. The term patient or subject includes human and veterinary subjects.

As used herein the terms treatment, treat, or treating refers to a method of reducing the effects of an injury or symptom of the injury. Thus in the disclosed method, treatment can refer to a 10%, 20%, 30%, 40%, 50%, 60%, 70%, 80%, 90%, or 100% reduction in the severity of an established injury or symptom of the injury. For example, a method for treating an injury is considered to be a treatment if there is a 10% reduction in one or more symptoms of the injury in a subject as compared to a control. Thus the reduction can be a 10%, 20%, 30%, 40%, 50%, 60%, 70%, 80%, 90%, 100%, or any percent reduction in between 10% and 100% as compared to native or control levels. One of skill in the art would be able to detect reduction in one or more symptoms of the injury through imaging of the injury (e.g. MRI), determination of levels of biomarkers (e.g., reduction in level of BACE1, presenilin-1, and/or amyloid precursor protein (APP)), and behavioral and cognitive tests (e.g., motor coordination test and spatial learning tests). It is understood that treatment does not necessarily refer to a cure or complete ablation of the injury or symptoms of the injury.

Prophylactic treatment reduces or eliminates the symptoms and/or delays the onset of symptoms, as compared to these in the absence of treatment. For example, the methods herein reduce the risk of Alzheimer's associated with traumatic brain injury (TBI), but can also reduce more acute effects such as short-term memory loss associated with TBI. Similarly, imaging and biomarker detection can show prophylactic effects.

As used herein, increase or decrease are used relative to a control value or condition, e.g., in the absence of treatment or before or after treatment effects. A test subject, optionally, is compared to a control group or control value.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus , if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Aβ and APP secretases levels increased following Traumatic Brain Injury (TBI)

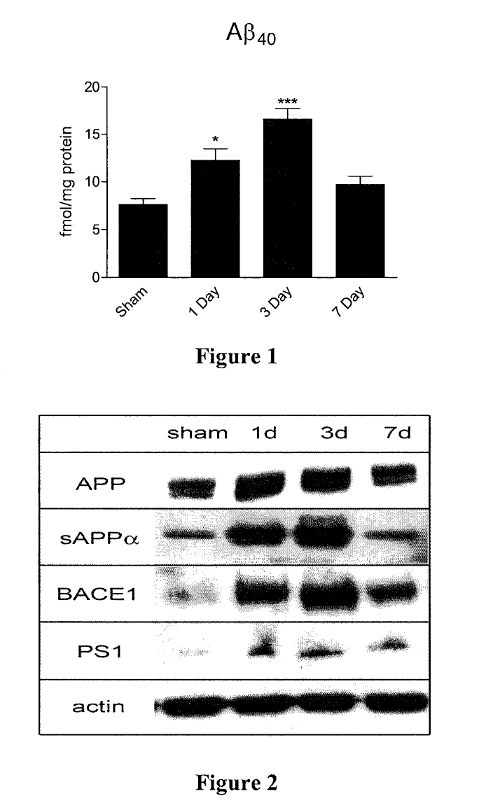

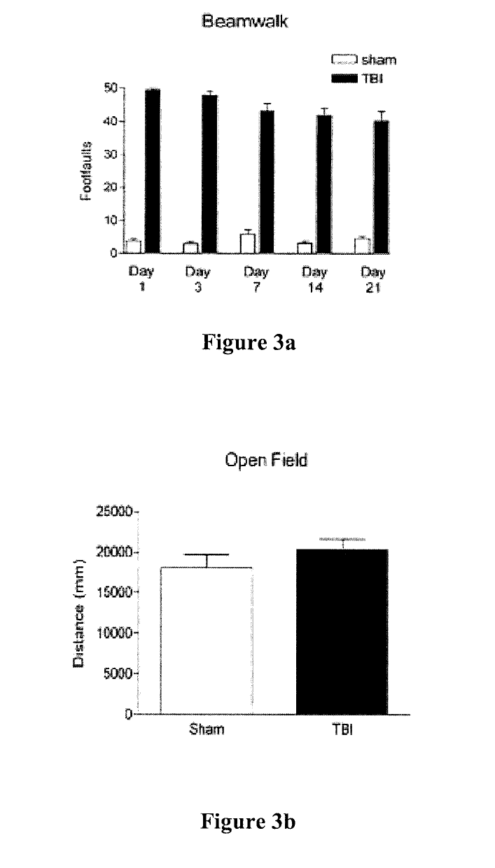

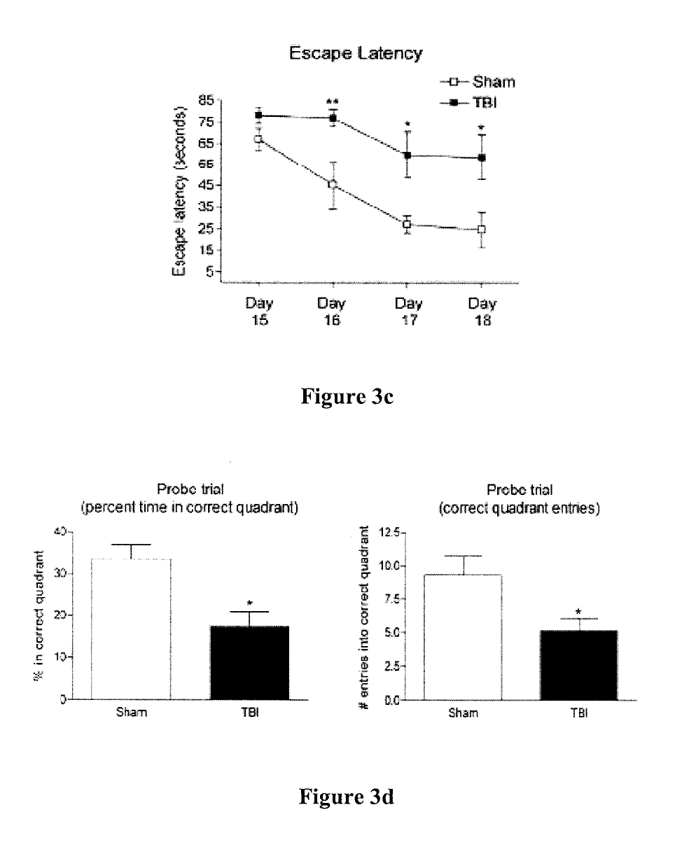

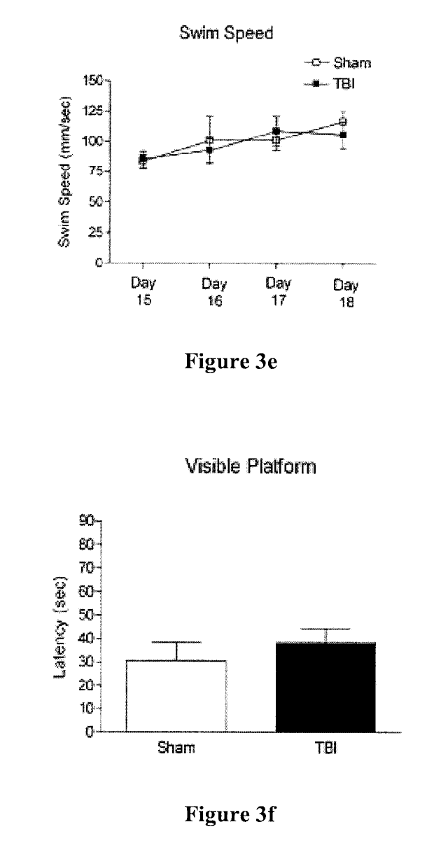

In initial experiments, Traumatic Brain Injury (TBI)-induced protein changes in a nontransgenic mouse were characterized. TBI was performed by controlled cortical impact (CCI) of the left parietal cortex. This model induces both necrotic and apoptotic cell death, causing brain lesion and the development of behavioral deficits (Fox et al., J. Neurotrauma 15:599-614 (1998)). It has recently been reported that interstitial fluid Aβ concentrations correlate with neurological function in the injured human brain, with Aβ accumulating as neurological function improved in the days after trauma (Brody et al., Science 321:1221-4 (2008)). Exposure to experimental TBI resulted in the accumulation of endogenous mouse Aβₓ₋₄₀ peptide in the ipsilateral cortex. Aβ levels increased by almost 120% at 3 days after injury before normalizing by 7 days (FIG. 1). The accumulation of Aβ corresponded with increased protein amounts of APP, Bace1, and presenilin-1 (FIG. 2), as has been previously reported in other animal models and humans (Chen et al., Am. J. Pathol. 165:357-71 (2004), Iwata et al., J. Neuropathol. Exp. Neurol. 61:1056-68 (2002), Blasko et al., J. Neural Transm. 111:523-36 (2004), Cribbs et al., Neuroreport 7:1773-6 (1996), Nadler et al., Glia 56:552-67 (2008), Uryu et al., Exp. Neurol. 208:185-92 (2007)). Soluble APP-α, which is purported to be neuroprotective (Thorton et al, Brain Res. 1094:38-46 (2006)), was also increased after injury. Functionally, this model of TBI causes deficits in fine motor coordination (beam walk test) (FIG. 3a) in the absence of gross motor deficits (open-field test) (FIG. 3b). Injured mice also have hippocampal deficits, with reduced spatial learning in a Morris water maze test (escape latency and probe trial)(FIGS. 3c and 3d). TBI did not cause any alterations in the ability of the mice to find a visual platform, nor did it affect the swim speed of the mice (FIGS. 3e and 3f).

### Example 2

### Improved Behavior and Reduced Hippocampal Cell Death Following TBI in BACE1 Knockout Mice

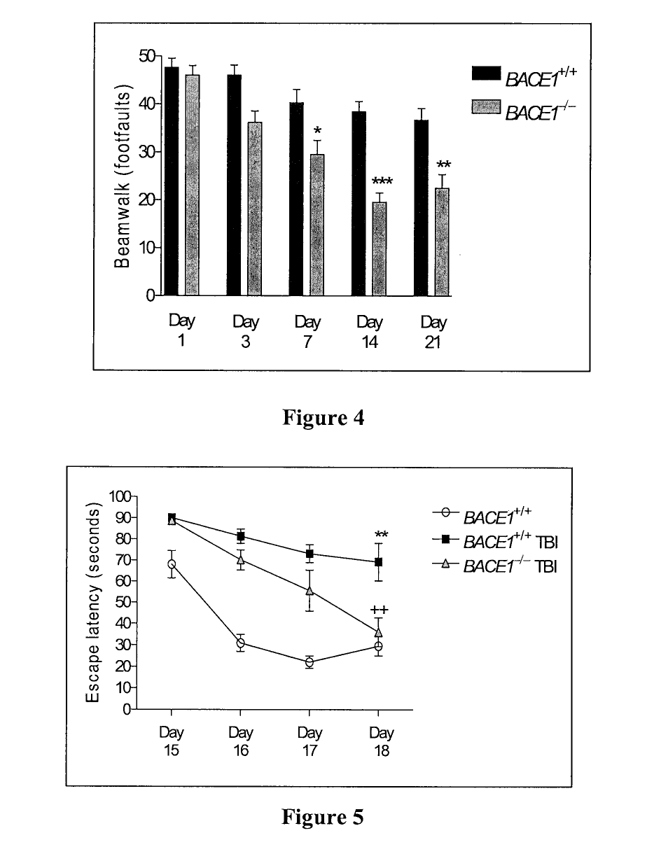

To test the effect of β-secretase on TBI outcome, the B-APP cleaving enzyme-1 (BACE1) Bace1^{-/-} knockout mouse was used. Bace1 is the initial rate-limiting enzyme for Aβ production. Bace1^{-/-} mice are unable to produce any species of Aβ (Cai et al., Nat. Neurosci. 4:233-4 (2001)), and the absence of Aβₓ₋₄₀ in brain homogenates by ELISA (0.1±0.09 fmol per mg protein) was confirmed. CCI surgery was performed, and the beam walk test was used to detect fine motor coordination aberrations (Fox et al., J. Neurotrauma 15:599-614 (1998)). This test examines the number of errors (foot faults) made by the right hind limb over 50 steps. The mice were trained on the test before injury, and the test was repeated on 1 day, 3 days, 7 days, 14 days, and 21 days after TBI. Injured Bace1^{-/-} mice made fewer foot faults at multiple time points, with a 38% final improvement over injured Bace1^{+/+} mice (FIG. 4, P<0.001).

A Morris water maze paradigm was used to assess spatial learning by training mice to locate a hidden, submerged platform using extramaze visual information (Fox et al., J. Neurotrauma 15:599-614 (1998)). The test was conducted 15-18 days after injury, and each mouse was tested for four trials per day for four consecutive days. Uninjured Bace1^{+/+} mice were used as a behavioral control. Injured Bace1^{+/+} mice had significant learning impairments on all 4 days of testing compared to uninjured Bace1^{+/+} mice (P<0.01). Injured Bace1^{-/-} mice, however, improved rapidly, and on the final day of testing were indistinguishable from uninjured mice (FIG. 5, P<0.01). All groups had similar escape latencies when presented with a visual platform probe test (uninjured Bace1^{+/+}, 23.4±6.3 seconds; injured Bace1^{+/+}, 21.1±7.8 seconds; injured Bace1^{-/-}, 21.2±4.9 seconds).

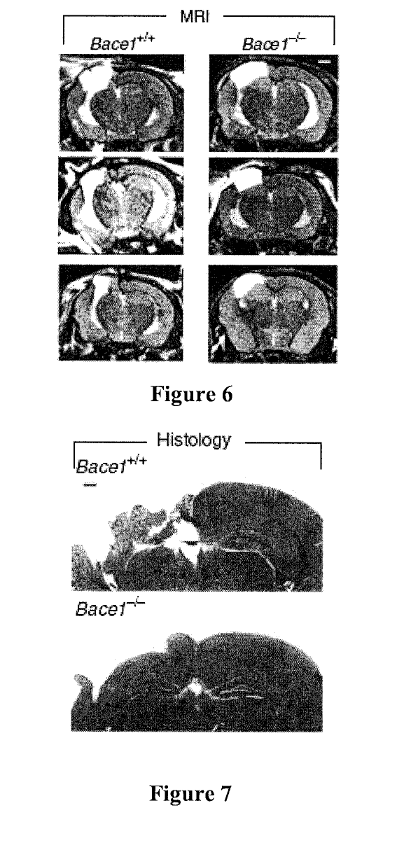

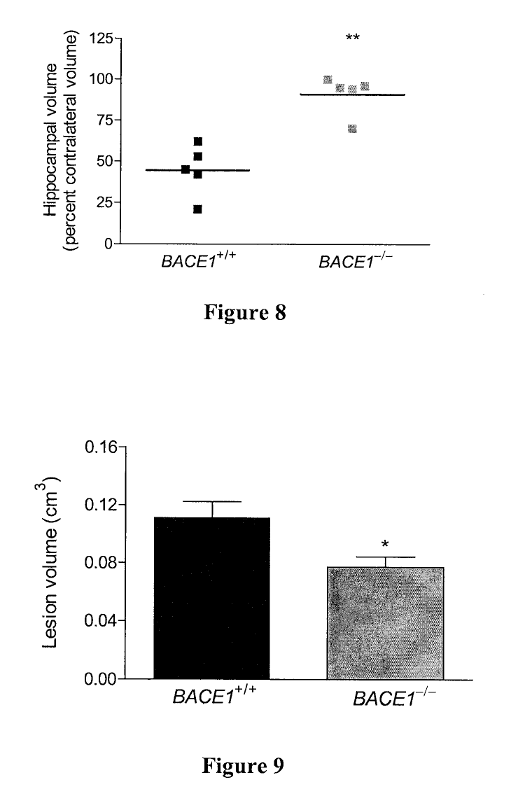

Five mice in each TBI group were randomly chosen for T2-weighted magnetic resonance imaging (MRI) analysis at 21 days after trauma. MRI assessment of damage in injured Bace1^{+/+} mice showed that the lesion was extensive, spreading from the cortex through the hippocampus and connecting to the lateral ventricles. However, injured Bace1^{-/-} mice showed considerable sparing of brain tissue, particularly of the ipsilateral hippocampus (FIG. 6). This was confirmed by fixed brain tissue sections (FIG. 7). Quantitative analyses showed that injured Bace1^{+/+} mice lost 65.4%±6.8% of the hippocampal tissue, compared to only 9.0%±5.3% for injured Bace1^{-/-} mice (FIG. 8, P<0.01). Total lesion volume was reduced by 30% in inured Bace1^{+/+} mice (FIG. 9, P<0.05).

### Example 3

### γ-Secretase Inhibition Improves Behavioral Outcome and Reduces Hippocampal Cell Death After TBI

The other secretase involved in amyloidogenic processing of APP is γ-secretase, and the pharmacological inhibitor N-[N-(3,5-difluorophenacetyl-_{L}-alanyl)]-S-phenlyglycine t-butyl ester (DAPT) was used to block enzyme activity. Previous work has shown that familial presenilin-1 mutations known to cause Alzheimer's disease increase Aβ levels by 30% (Duff et al., Nature 383:710-13 (1996)), suggesting that this level of inhibition would be sufficient to prevent a disease state. DAPT was administered orally at 30 mg per kg body weight, a dose that has previously been shown to reduce Aβ levels in vivo (Duff et al., Nature 383:710-3 (1996)). Each mouse received the initial dose 15 minutes after CCI or sham surgery and then twice daily for 21 days. This dosing regime was implemented because Aβ is known to drop after γ-secretase inhibition before recovering over several hours (Abramowski et al., J. Pharmacol. Exp. Ther. 327:411-24 (2008), El Mouedden et al., Curr. Pharm. Des. 12:671-6 (2006)). To confirm γ-secretase inhibition in DAPT-treated mice, Aβ concentrations were measured. Three hours after final drug administration, Aβ levels were reduced by 25% (13.76±0.8 fmol per mg protein (vehicle-treated TBI mice) versus 10.42±0.4 fmol per mg protein (DAPT-treated TBI mice); P<0.01), and APP carboxy-terminal fragments accumulated.

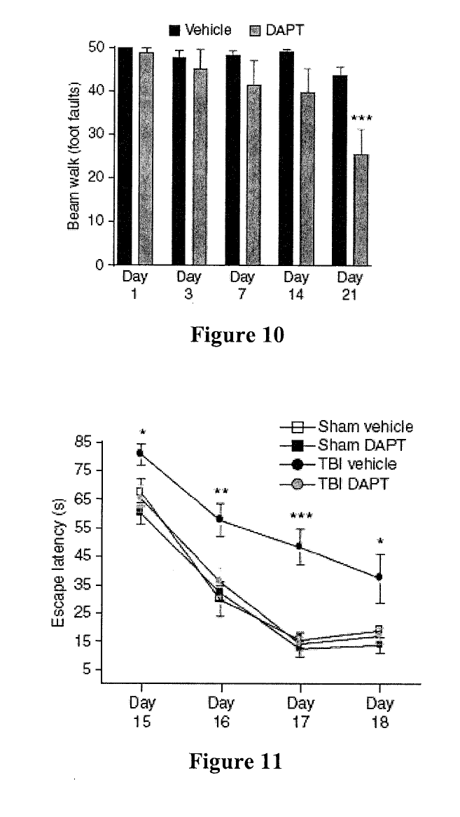

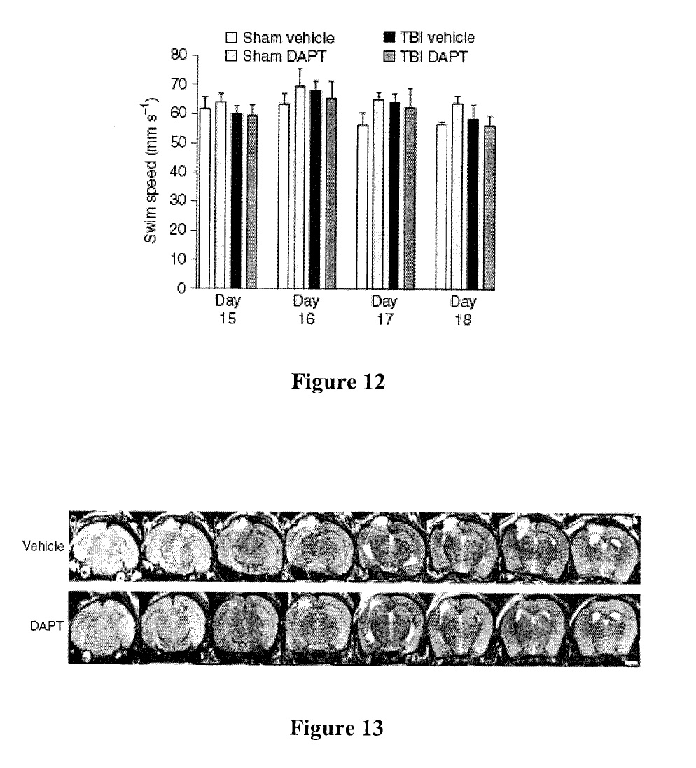

On the day after CCI, the first beam walk test was conducted. Vehicle-treated TBI mice made 50±0 foot faults and improved to 43±2 foot faults on the final day of testing (FIG. 9). DAPT-treated TBI mice had a foot fault rate of 48±1 on the day after surgery; however, by 21 days after injury, they averaged 25±5 foot faults, an improvement of 42% over vehicle-treated TBI mice (FIG. 10, P<0.001). Spatial learning with the Morris water maze was also tested. Vehicle-treated TBI mice had learning impairments on all 4 days of the test, whereas the behavior of DAPT-treated TBI mice was indistinguishable from sham-injured mice (FIG. 11). All mice had similar swim speeds in the maze (FIG. 12), and similar escape latencies when presented with a visible platform probe (vehicle-treated, sham-operated mice, 15.5±3.1 seconds; DAPT-treated, sham-operated mice, 13.6±3.4 seconds; vehicle-treated TBI mice, 15.1±2.6 seconds; DAPT-treated TBI mice 17.5±3.7 seconds).

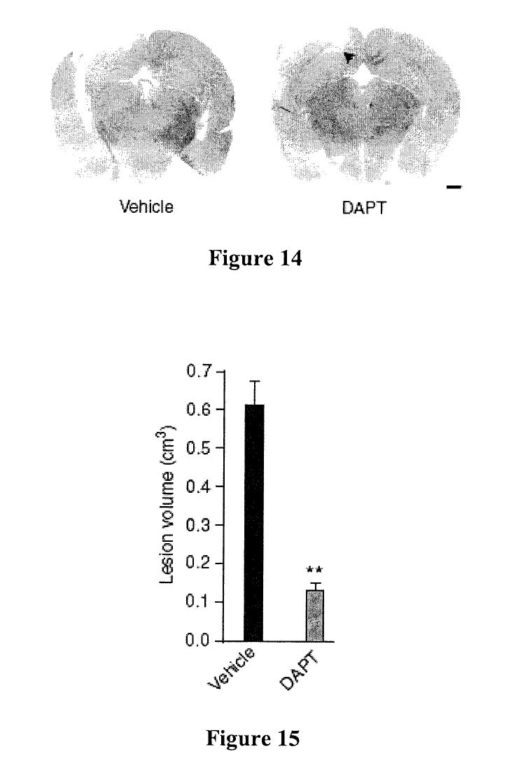

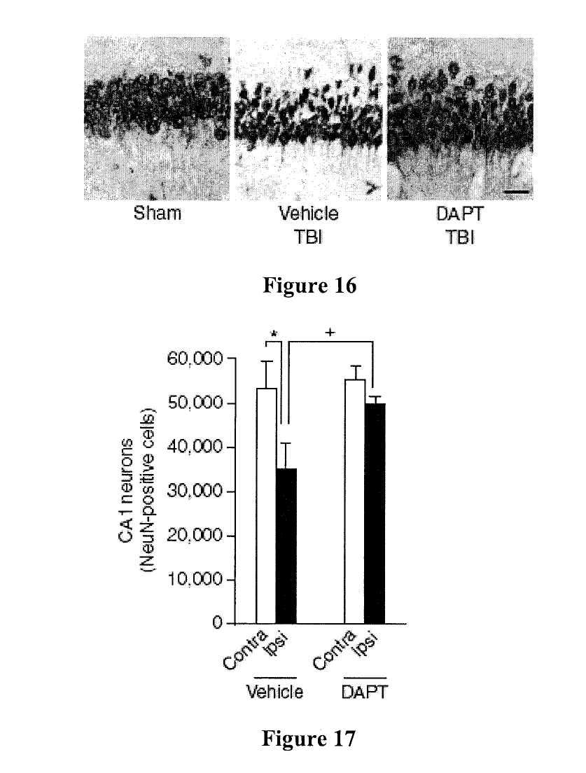

MRI revealed that DAPT treatment substantially reduced lesion size in injured mice (FIG. 13). The representative MRI images show a vehicle-treated TBI mouse with the brain lesion visible through 4 mm (8 mm×0.5 mm longitudinal sections) of tissue, whereas DAPT treatment results in damage through only 1.5 mm (3 mm×0.5 mm sections) (FIG. 13). Histological assessment showed that DAPT treatment spared large areas of cortex and subcortical white matter tracts, notably the corpus callosum (FIG. 14), and decreased lesion volume by >70% (FIG. 15, P<0.01). The damage to the hippocampus was not as extensive as that seen in the Bacel study; however, cell loss in the CA1 region of mice after TBI was clearly visible (FIG. 16). This allowed quantification of cell loss in the hippocampus (FIG. 17). Neurons were stained with NeuN, a neuronal marker, and an unbiased stereology count was performed. TBI caused a 34% decrease in NeuN-positive cells in the CA1 region of the ipsilateral hippocampus (P<0.05). DAPT treatment attenuated this cell loss, resulting in a 10% reduction in CA1 neurons (P<0.05 versus vehicle-treated TBI mice). Thus, DAPT treatment decreased cell death in hippocampal neurons, reduced cortical cell loss and reduced white matter damage in the corpus callosum. These findings are supported by data from a mouse model of stroke, in which DAPT treatment reduced infarct area and improved neurological function after ischemia and reperfusion (Arumugam et al., Nat. Med. 12:621-3 (2006)).

The temporal dissociation between the peak of APP secretase protein abundance (day 3) and the learning improvements (day 18) suggest that the APP secretases have a detrimental role in the initiation of secondary injury, rather than continually producing toxic factor. By decreasing secretase activity during this crucial period, the ongoing damage of secondary injury can be blocked. Further experiments, such as Aβ rescue in Bace1-knockout mice, would test the role of Aβ after TBI; however, the data demonstrate that the APP secretases are key mediators of tissue loss after injury. Knockout of the Bace1 gene has been shown to decrease cell loss and behavioral deficits in a classically used TBI mouse model. Similarly, pharmacological inhibition of γ-secretase activity reduces post-traumatic tissue loss and improves motor and cognitive recovery. Notably, the γ-secretase inhibitor was administered after trauma, modeling a clinically relevant situation. Together, these studies suggest that modulation of APP secretases may provide new therapeutic targets for the treatment of TBI.

### Example 4

### A Pharmacological BACE1 Inhibitor Prevents Injury Induced Aβ

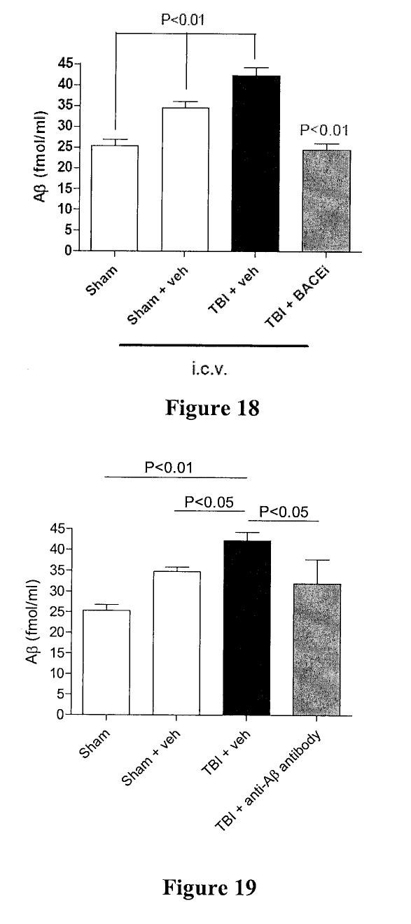

A BACE1 inhibitor (BACE inhibitor IV) can prevent the spike in Aβ induced by injury. Aβ levels were measured in sham injured mice, sham injured mice treated with vehicle, TBI mice treated with vehicle, and TBI mice treated with BACE inhibitor IV. Vehicle (40% DMSO in saline) and inhibitor were administered by icy injection into the ipsilateral cerebral ventricle (5 μ1 over 5 minutes) 15 minutes after TBI or sham surgery, and Aβ levels in the ipsilateral cortex were determined by ELISA. Vehicle administration via icy injection in sham injured mice caused a an increase in Aβ production compared to sham injured mice alone (36%, P<0.01). TBI further increased Aβ levels (24%, P<0.01 compared to sham vehicle). BACE inhibitor attenuated the increase in Aβ caused by either icy injection or TBI (P<0.01 vs. sham-vehicle or TBI-vehicle) (FIG. 18).

### Example 5

### Administration of Anti-Aβ Antibody Prevents TBI-Induced Increase in Aβ

In order to test the theory that anti-Aβ antibodies could be used to reduce Aβ levels following injury, we injected an anti-Aβ antibody into the ipsilateral cerebral ventricle on mice following TBI. Mice were euthanized 24 hours after injury and levels of Aβ in the ipsilateral cortex were measured by ELISA. It was found that icy injection of vehicle alone increased Aβ compared to sham injured mice with no icy injection (P<0.01) and TBI with vehicle further increased Aβ levels; however, icy injection of anti-Aβ antibody attenuated these effects (FIG. 19, P<0.05).

### Example 6

### Determining the Therapeutic Window for γ-Secretase Inhibition

DAPT (30 mg/kg p.o.) is administered 15 minutes after surgery and every 12 hours for 1, 3, 7, 14 or 21 days (n=10). Fine motor coordination deficits are quantified using the beam walk test on days 1, 3, 7, 14, and 21. Spatial learning and memory are tested using a Morris water maze on days 15-19. MRI is conducted on day 21 to assess lesion volume. Brain tissue is kept for histological assessment of grey and white matter damage.

In order to determine the effect of DAPT on behavioral and/or histological outcome after TBI, DAPT (30 mg/kg p.o.) is administered at one of the following timepoints: 15 minutes, 30 minutes, 1 hour, 3 hours, 6 hours, 12 hours, or 24 hours post-trauma (n=10). Fine motor coordination deficits are quantified using the beam walk test on days 1, 3, 7, 14, and 21. Spatial learning and memory are tested using a Morris water maze on days 15-19. MRI is conducted on day 21 to assess lesion volume. Brain tissue is kept for histological assessment of grey and white matter damage.

What is claimed is:

1. A method of treating an acute central nervous system injury in a subject comprising administering to the subject a γ or β secretase inhibitor, wherein the γ or β secretase inhibitor is administered to the subject within 72 hours of acute central nervous system injury.

2. The method of claim 1 further comprising selecting a subject with a central nervous system injury or at risk of a central nervous system injury.

3. The method of claim 1, wherein the γ or β secretase inhibitor is administered on the day of injury.

4. The method of claim 3, wherein the γ or β secretase inhibitor is administered within 1 hour of injury.

5. The method of claim 4, wherein the γ or β secretase inhibitor is administered within 15 minutes of injury.

6. The method of claim 1, wherein the injury is selected from the group consisting of a traumatic brain injury, a cerebrovascular event, a spinal cord injury, and a central nervous system surgery.

7. The method of claim 6, wherein the injury is a central nervous system surgery and wherein the γ or β secretase inhibitor is administered before, during, or after surgery or is administered in any combination of before, during, or after surgery.

8. The method of claim 1, wherein the γ or β secretase inhibitor is administered to the subject at least once daily for 21 days or less.

9. The method of claim 8, wherein the γ or β secretase inhibitor is administered to the subject at least twice daily.

10. The method of claim 8, wherein the γ or β secretase inhibitor is administered for at least three days.

11. The method of claim 1, wherein the secretase inhibitor is a γ secretase inhibitor.

12. The method of claim 11, wherein the γ secretase inhibitor is N-[N-(3,5-Difluorophenacetyl)-L-alanyl]-S-phenylglycine t-butyl ester (DAPT) or a derivative thereof.

13. The method of claim 12, wherein the DAPT or derivative thereof is administered in a dose of 10-100 mg/kg up to twice daily.

14. The method of claim 1, wherein the γ secretase inhibitor is LY450139 or a derivative thereof.

15. The method of claim 14, wherein the LY450139 or derivative thereof is administered in a dose of 10-100 mg/kg up to twice daily.

16. The method of claim 1, wherein the secretase inhibitor is a β secretase inhibitor.

17. The method of claim 16, wherein the β secretase inhibitor is BACE inhibitor IV or a derivative thereof.

18. A method of treating an acute central nervous system injury in a subject comprising administering to the subject an Aβ antibody, wherein the Aβ antibody is administered to the subject within 72 hours of acute central nervous system injury.

19. The method of claim 18 further comprising selecting a subject with a central nervous system injury or at risk of a central nervous system injury.

20. The method of claim 18, wherein the Aβ antibody is administered on the day of injury.

21. The method of claim 18, wherein the Aβ antibody is administered within 1 hour of injury.

22. The method of claim 20, wherein the Aβ antibody is administered within 15 minutes of injury.

23. The method of claim 18, wherein the injury is selected from the group consisting of a traumatic brain injury, a cerebrovascular event, a spinal cord injury, and a central nervous system surgery.

24. The method of claim 23, wherein the injury is a central nervous system surgery and wherein the Aβ antibody is administered before, during, or after surgery or administered in any combination of before, during, or after surgery.

25. The method of claim 18, wherein the Aβ antibody is administered to the subject at least once daily for 21 days or less.

26. The method of claim 25, wherein the Aβ antibody is administered to the subject at least twice daily.

27. The method of claim 25, wherein the Aβ antibody is administered for at least three days.

28. A method of treating an acute central nervous system injury in a subject comprising administering to the subject an immunogenic fragment of Aβ, wherein the immunogenic fragment of Aβ is administered to the subject within 24 hours of acute central nervous system injury.

29. An auto-injector for emergency treatment of an acute central nervous system injury in a subject, wherein the auto-injector comprises a γ or β secretase inhibitor.

30. The auto-injector of claim 29, wherein the secretase inhibitor is a γ secretase.

31. The auto-injector of claim 30, wherein the γ secretase inhibitor is DAPT or a derivative thereof.

32. The auto-injector of claim 29, wherein the secretase inhibitor is a β secretase inhibitor.

33. The auto-injector of claim 32, wherein the β secretase inhibitor is BACE inhibitor IV or a derivative thereof.

34. A kit comprising the auto-injector of claim 29.
